(19) United States
(12) Patent Application Publication
(10) Pub. No.: US 2017/0065454 A1
(43) Pub. Date: Mar. 9, 2017

(54) An Eye-Dropper Positioning Device, a Method for Delivering an Eye-Drop and an Eye Examination Device

(71) Applicant: Frank Anthony TEDESCO, Church Point, NSW (AU)

(72) Inventor: Frank Anthony Tedesco, New South Wales (AU)

(21) Appl. No.: 15/121,995

(22) PCT Filed: Feb. 11, 2015
(86) PCT No.: PCT/AU2015/000075

§ 371 Date: Aug. 26, 2016

(30) Foreign Application Priority Data
Feb. 28, 2014 (AU) 2014900659

(51) Int. Cl. A61F 9/00

(52) U.S. Cl. CPC A61F 9/0026

(57) ABSTRACT

There is disclosed an eye-dropper positioning device (10) including a container (12) having at least a substantially transparent portion and defining an interior. The device (10) further includes a fluid (14) at least partially filling the interior of the container (12). The device (10) further includes a mirror assembly (16) located in the interior of the container (10). The mirror assembly (16) comprises at least one reflecting surface (20); and an orientating means (22) buoyantly supported by the fluid (14) and configured to substantially face the at least one reflecting surface (20) downwardly such that an image of an eye under the container (10) is reflected by the at least one reflecting surface (20) back to the eye through the substantially transparent portion of the container (10).

### FIELD

The present invention relates to an eye-dropper positioning device, a method for delivering an eye-drop, and an eye examination device.

### BACKGROUND

Eye-drops are a saline-based solution which is administrated to an eye for lubrication and/or delivery of medication. Typically, eye-drops are sold contained in an eye-dropper. The eye-dropper is often designed with a nozzle that allows eye-drops to be dispensed therefrom upon squeezing of the eye-dropper.

In order to self-administer eye-drops, a common known method is for a person to tilt their face upwards and position the eye-dropper above and close to their eye. The person will then aim the nozzle directly at their eye and then squeeze the eye-dropper until a desired quantity of eye-drops are dispensed from nozzle into their eye.

A disadvantage of the above known method is that it is not always possible to accurately aim the nozzle directly at the eye, particularly in the case of the elderly or disabled. This inaccuracy can lead to wastage of the eye-drops, and there is a risk that inaccurately dispensed eye-drops may firstly land on surrounding skin, which may be harbouring bacteria, and thereby become contaminated prior to entering the eye. Further, inaccurately dispensed eye-drops can lead to the person missing a prescribed dose of medication and thereby cause the failure of treatment. Still further, without accurate aiming of the nozzle, administration of eye-drops to specific areas (e.g. the pupil or the white) of the eye is practically impossible.

Due to the issues involved in the above known methods, a person who requires the administration of eye-drops will often seek the aid of others. However, it is not always possible to find another person for assistance. Further, it is not always feasible or affordable to hire a health care professional (e.g. a nurse) to aid in the administration of eye-drops, particularly as eye-drops need to be administered on a regular basis.

### Object of Invention

It is the object of the present invention to substantially overcome or ameliorate one or more of the above disadvantages, or at least provide a useful alternative.

### SUMMARY OF INVENTION

There is disclosed herein an eye-dropper positioning device including:

a container having at least a substantially transparent portion and defining an interior;

a fluid at least partially filling the interior of the container; and

a mirror assembly located in the interior of the container, the mirror assembly comprising:
at least one reflecting surface; and
an orientating means buoyantly supported by the fluid and configured to substantially face the at least one reflecting surface downwardly such that an image of an eye under the container is reflected by the at least one reflecting surface back to the eye through the substantially transparent portion of the container.

The orientating means is preferably located above the at least one reflecting surface.

The orientating means preferably comprises a material buoyant within the fluid. The material preferably comprises cork.

The orientating means preferably comprises an air-filled chamber.

The container is preferably substantially shaped as a sphere.

The interior and the mirror assembly preferably each define a diameter, the diameter of the mirror assembly being less than the diameter of the interior.

The substantially transparent portion of the container and/or the fluid preferably magnifies the image of the eye.

The substantially transparent portion of the container is preferably formed from polycarbonate.

The substantially transparent portion of the container is preferably formed from polypropylene.

The fluid is preferably of a high viscosity.

The fluid preferably comprises liquid silicone.

The fluid is preferably substantially transparent.

The eye-dropper positioning device preferably further comprises an attachment mechanism configured to allow attachment of a swivel connector to the container.

The at least one surface is preferably substantially planar. The at least one reflecting surface is preferably substantially outwardly convex. The at least one reflecting surface is preferably substantially concave. The orientating means is preferably further configured to substantially horizontally level at least an outer periphery of the at least one reflecting surface.

There is also disclosed herein a method for delivering an eye-drop from an eye-dropper to an eye, the method comprising:

positioning a mirror assembly having at least one reflecting surface above the eye;

facing the at least one reflecting surface downwardly such that an image of the eye is reflected back to the eye;

positioning an eye-dropper using the reflected image of the eye such that a nozzle of the eye-dropper is located between the at least one reflecting surface and the eye, and the nozzle is aimed at a desired portion of the eye; and

actuating the eye-dropper to deliver the eye-drop to the desired portion of the eye.

The mirror assembly preferably comprises an orientating means, and wherein the step of facing the at least one reflecting surface downwardly comprises buoyantly supporting the orientating means of the mirror assembly in a fluid. The orientating means preferably comprises a buoyant material. The fluid is preferably of a high viscosity.

The method preferably further comprises magnifying the reflected image of the eye prior to being reflected back to the eye.

The desired portion is preferably the pupil of the eye.

The desired portion is preferably the white of the eye.

The at least one reflecting surface is preferably substantially planar. The at least one reflecting surface is preferably substantially outwardly convex. The at least one reflecting surface is preferably substantially concave. The step of facing the at least one reflecting surface downwardly comprises horizontally levelling at least an outer periphery of the at least one reflecting surface.

There is also disclosed an eye examination device including:

a container having at least a substantially transparent portion and defining an interior;

a fluid at least partially filling the interior of the container; and

a mirror assembly located in the interior of the container, the mirror assembly comprising:
at least one reflecting surface; and
an orientating means buoyantly supported by the fluid and configured to substantially face the at least one reflecting surface downwardly such that an image of an eye under the container is reflected by the at least one reflecting surface back to the eye through the substantially transparent portion of the container.

### BRIEF DESCRIPTION OF DRAWINGS

Preferred embodiments of the invention will be described hereinafter, by way of examples only, with reference to the accompanying drawings, wherein:

FIG. 1 is a front view of a first embodiment of an eye-dropper positioning device;

FIG. 2 is a front view of the eye-dropper positioning device of FIG. 1 positioned above an eye with an eye-dropper positioned between the eye-dropper positioning device and the eye;

FIG. 3 is a perspective view of the eye-dropper positioning device of FIG. 1 held by a hand;

FIG. 4 is an enlarged front view of a second embodiment of an eye-dropper positioning device;

FIG. 5 is a front view of a third embodiment of an eye-dropper positioning device with an attachment mechanism protruding from a side of the container;

FIG. 6 is a front view of a fourth embodiment of an eye-dropper positioning device with an attachment mechanism protruding from a top of the container;

FIG. 7 is a perspective view of a fifth embodiment of an eye-dropper positioning device held by a hand;

FIG. 8 is a front view of the eye-dropper positioning device of FIG. 7 positioned above an eye with an eye-dropper positioned between the eye-dropper positioning device and the eye;

FIG. 9 is a front view of the eye-dropper positioning device of FIG. 7 with a handle attached thereto;

FIG. 10 is a front view of a sixth embodiment of an eye-dropper device;

FIG. 11 is a top view of the eye-dropper device of FIG. 10;

FIG. 12 is a front view of a seventh embodiment of an eye-dropper device;

FIG. 13 is an enlarged fragmentary cross-sectional view of the eye-dropper device of FIG. 12; and

FIG. 14 is a top view of the eye-dropper device of FIG. 12.

### DESCRIPTION OF EMBODIMENTS

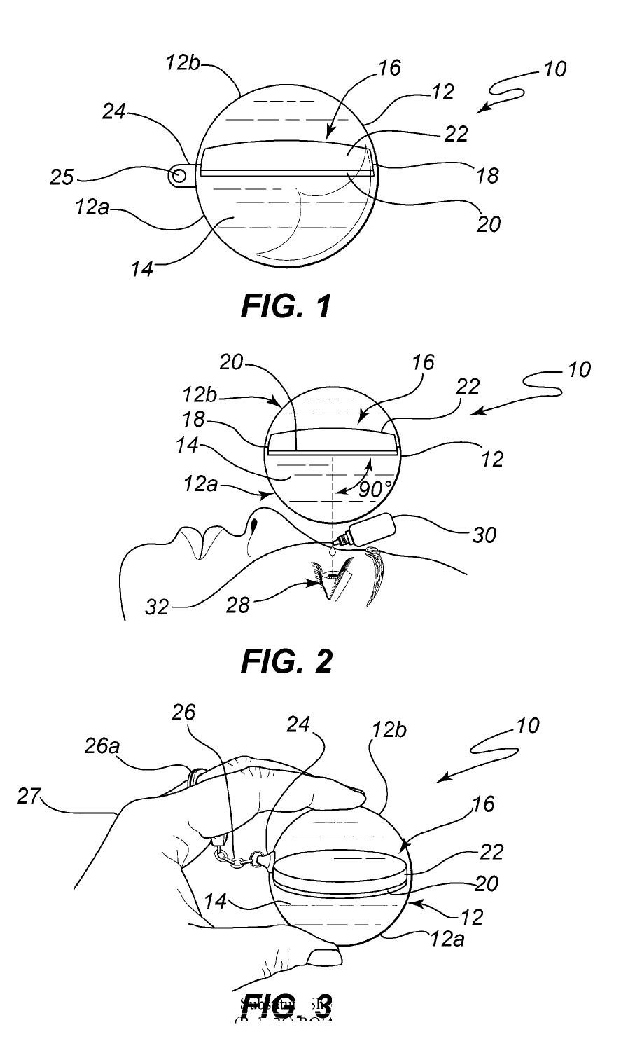

FIGS. 1 to 3 show a first embodiment of an eye-dropper positioning device 10. The eye-dropper positioning device 10 includes a container 12, a fluid 14 and a mirror assembly 16. The container 12 is spherical in shape and defines a fluid-tight spherical interior. The container 12 is substantially transparent and is formed from two semi-spherical pieces of high transparency clear polypropylene 12a, 12b that are glued or heat welded together along a seam 18. It will be appreciated that, in other embodiments, the container 12 is formed from two semi-spherical pieces of high transparency clear polycarbonate.

The fluid 14 is a liquid silicone such as, for example, Dow Corning® 200 Fluid 1000 CST. This liquid is of a high viscosity and is substantially transparent. The interior of the container 12 is partly filled with the fluid 14.

The mirror assembly 16 is located in the interior of the container 12 and suspended in the fluid 14. The mirror assembly 16 has a diametric periphery that is slightly less than a diameter of the interior of the container 12. This allows the mirror assembly 16 to rotate within the interior of the container 12 without obstruction. The mirror assembly 16 comprises a planar silvered mirror 20 with a bottom reflecting surface and an orientating means 22. The planar mirror 20 and is located on a substantially flat underside of the mirror assembly 16.

The orientating means 22 is formed from cork which is buoyantly supported by the fluid 14 and is located above the planar mirror 20. The combination of the buoyancy and the location allows the orientating means 22 to substantially face the reflecting surface of the planar mirror 20 downwardly and horizontally level the planar mirror 20, irrespective of the orientation of the container 12, such that the reflecting surface of the planar mirror 20 is able to reflect images of objects located underneath.

The fluid 14 and the curvature of the container 12 allow images reflected from the reflecting surface of the planar mirror 20 to be refracted such that the reflected images are substantially magnified when seen from outside the container 12.

The eye-dropper positioning device 10 further includes an attachment mechanism 24 which sidewardly protrudes from the container 12. The attachment mechanism 24 is configured to allow attachment of a swivel connector 26 to the container 12. The attachment mechanism 24 has an aperture 25, extending from the front to the back, to receive the swivel connector 26 therethrough, to which may be connected a finger ring 26a or the like.

A typical use and operation of the eye-dropper positioning device 10 will now be described.

Referring to FIG. 3, the eye-dropper positioning device 10 is firstly held by a hand 27 of a user with the ring 26a on one of the user's fingers. Referring to FIG. 2, the user then upwardly tilts their face and looks directly above. The user then positions the eye-dropper positioning device 10 directly above and close to an eye 28 which requires the administration of eye-drops. As the eye-dropper positioning device 10 is positioned, the orientating means 22 will automatically substantially horizontally level the planar mirror 20 such that the image of the eye 28 is reflected by the reflecting surface of the planar mirror 20 back to the eye through the fluid 14 and the container 12. The user then adjusts the eye-dropper positioning device 10 until the reflected image of the eye 28 is focused and undistorted. The reflected image of the eye 28 is refracted and thereby magnified as it passes through the fluid 14 and the container 12. Accordingly, the user is able to see a magnified image of the eye 28.

Subsequently, with the other hand of the user, an eye-dropper 30 containing eye-drops is positioned such that a nozzle 32 of the eye-dropper 30 is located between the eye-dropper positioning device 10 and the eye 28. As the user is able to see a magnified image of the eye 28, the user is able to accurately aim the nozzle 32 at a desired portion of the eye 28 such as, for example, the pupil or the white. Once the nozzle 32 is aimed, the user will then squeeze the eye-dropper such that one or more eye-drops are dispensed from the nozzle 32 directly into the desired portion of the eye 28.

An advantage of the eye-dropper positioning device 10 is that a (magnified) image of the eye is provided such that eye-drops can be easily self-administered in an accurate manner. Accordingly, there is less wastage of eye-drops and there is reduced risk of the eye-drops becoming contaminated prior to entering the eye as the eye-drops can be easily administered directly into the eye (e.g. without prior contact with the surrounding skin).

Another advantage of the eye-dropper positioning device 10 is that the reflecting surface of the planar mirror 20 will always be able to provide a reflected image of the eye 28 to the user regardless of the orientation of the container 12

Another advantage of the eye-dropper positioning device 10 is that eye-drops can be administered to specific portions of the eye. This is especially beneficially when specific portions (e.g. the pupil or the white) of the eye need to be lubricated or medicated.

Another advantage of the eye-dropper positioning device 10 is that there is no need for assistance from another person to administer eye-drops to oneself.

Another advantage of the eye-dropper positioning device 10 is that most types of eye dropper 30 can be utilised, irrespective of size and brand.

Other advantages of the eye-dropper positioning device 10 are that it is easy to use, portable to carry, relatively inexpensive to manufacture, and there is no or minimal maintenance involved.

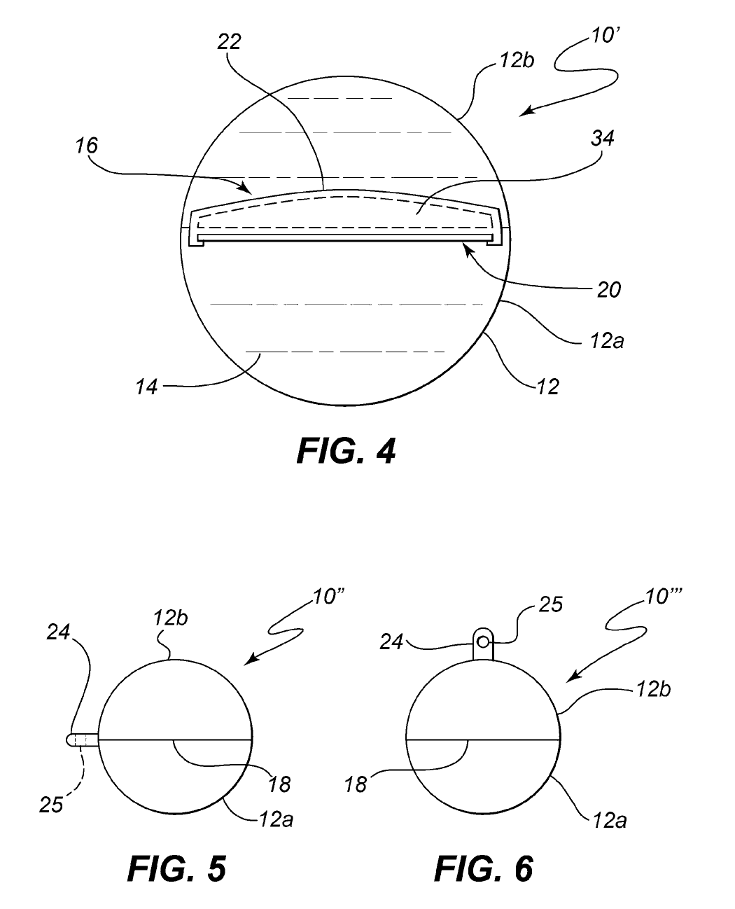

FIG. 4 shows an enlarged front view of a second embodiment of an eye-dropper positioning device 10′, and in which like reference numerals are used to denote like features to that of the eye-dropper positioning device 10. In this embodiment, the orientating means 22 of the eye-dropper positioning device 10′ is not formed from cork but instead comprises an air-tight plastic chamber 34 located above the planar mirror 20. The chamber 34 is filled with air such that the chamber 34 is buoyantly supported by the fluid 14.

FIG. 5 shows a third embodiment of an eye-dropper positioning device 10″, and in which like reference numerals are used to denote like features to that of the eye-dropper positioning device 10. In this embodiment, the attachment mechanism 24 of the eye-dropper positioning device 10″ sidewardly protrudes from the container 12 from the seam 18. The aperture 25 extends from the bottom to the top.

FIG. 6 shows a fourth embodiment of an eye-dropper positioning device 10′, and in which like reference numerals are used to denote like features to that of the eye-dropper positioning device 10. In this embodiment, the attachment mechanism 24 of the eye-dropper positioning device 10′ upwardly protrudes from the piece 12b of the container 12. The aperture 25 extends from the front to the back.

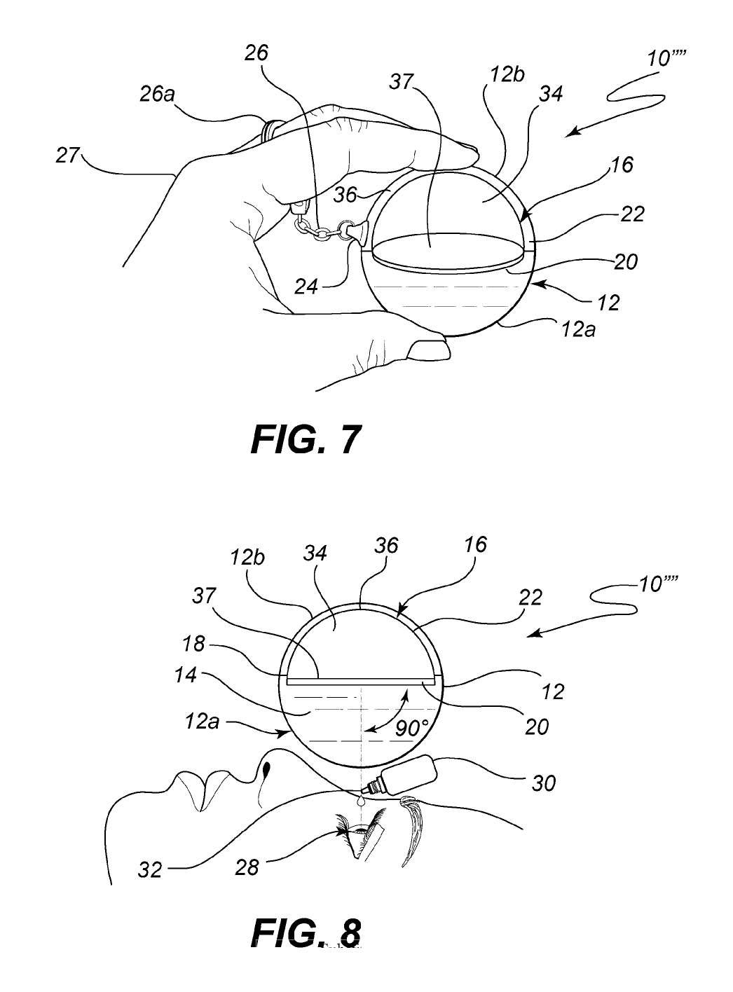

FIGS. 7 and 8 show a fifth embodiment of an eye-dropper positioning device 10″″, and in which like reference numerals are used to denote like features to that of the eye-dropper positioning device 10′. In this embodiment, the air-tight chamber 34 of the orientating means 22 is defined between a semi-spherical clear plastic wall 36 and a top non-reflective surface 37 of the planar mirror 20.

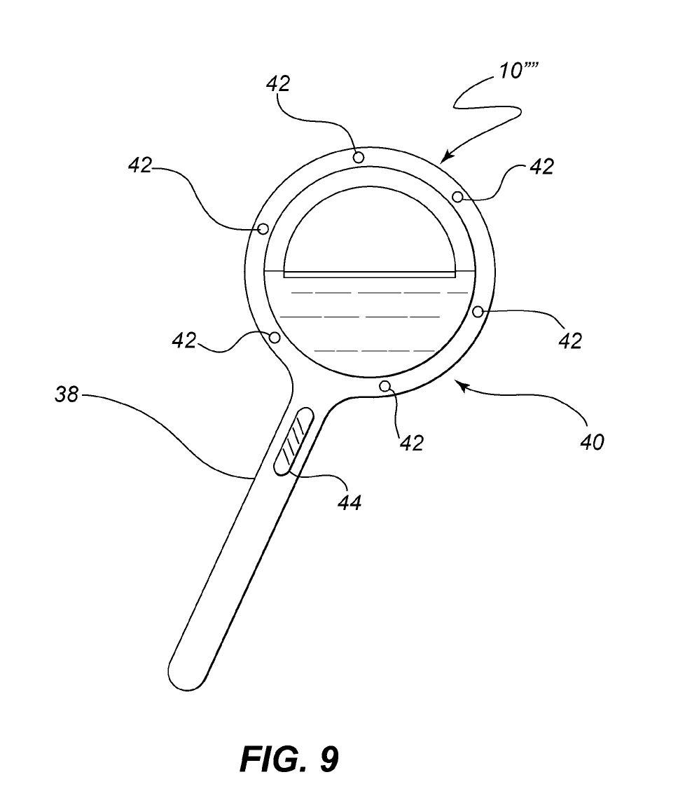

FIG. 9 shows the eye-dropper positioning device 10″ attached to a handle 38. The handle 38 comprises an engagement end in the form of a circular ring 40. The ring 40 is configured to fittingly receive the eye-dropper positioning device 10″ such that the eye-dropper positioning device 10″″ is secured therein. The handle 38 further comprises six LED lights 42 equally spaced around the ring 40 and are turned on/off by a button 44. It will be appreciated that the handle 38 allows the device 10″″ to be more easily controlled and positioned by the user. Further, it will be appreciated that the lights 42 allow eye-drops to be administered at night and/or in low-light environments.

In other embodiments, the inner diameter of the ring 40 may be adjustable to easily secure around the device 10″″ or release the device 10″″ already secured therein.

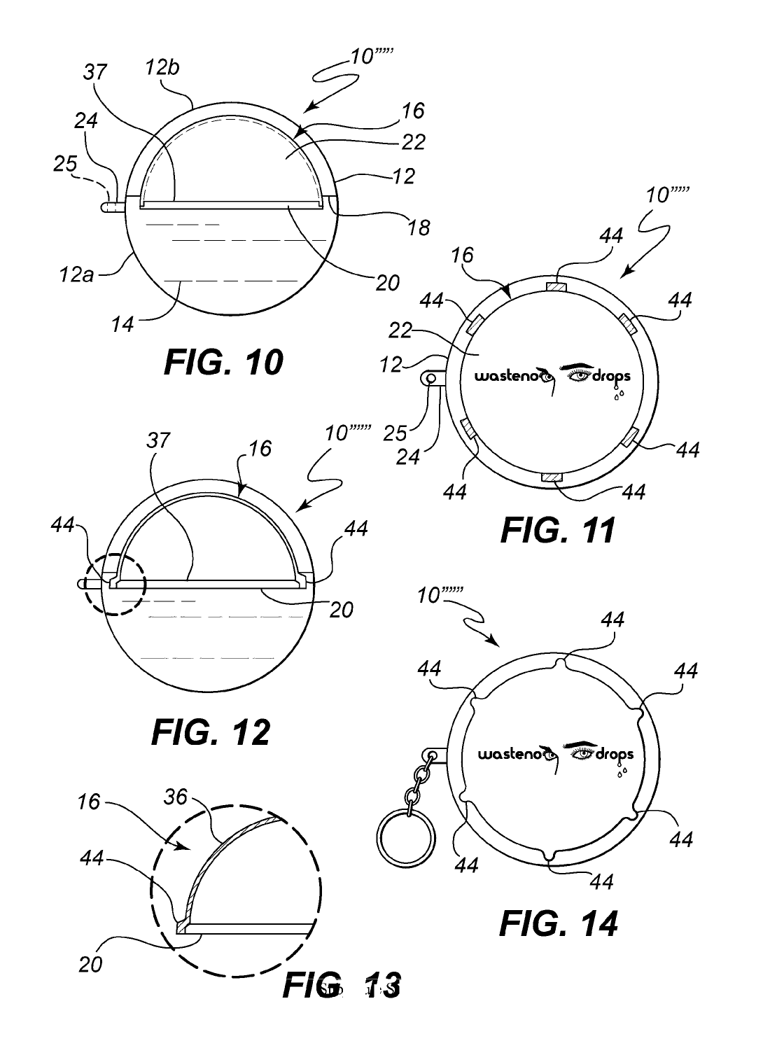

FIGS. 10 and 11 show a sixth embodiment of an eye-dropper positioning device 10′″″, and in which like reference numerals are used to denote like features to that of the eye-dropper positioning device 10″″. In this embodiment, the device 10′″″ comprises six spacers 44 glued to and located evenly around the diametric periphery of the planar mirror 20. The spacers 44 allow the mirror assembly 16 to rotate within the interior of the container 12 in a quick and smooth manner. As best seen in FIG. 11, the device 10′″″ also has information (e.g. advertisements, contact details etc) attached or printed on the top surface 37 of the planar mirror 20. As the planar mirror 20 is horizontally levelled by the orientating means 22, the information is able to be viewed from the top through the hemispherical clear plastic wall 36. It will be appreciated that other items may be attached to the top surface 37 of the planar mirror 20 for display, such as a compass, a small figurine, a miniature 3D eye-drop bottle or the like.

FIGS. 12 to 14 show a seventh embodiment of an eye-dropper positioning device 10″″″, and in which like reference numerals are used to denote like features to that of the eye-dropper positioning device 10″″″. In this embodiment, as best seen in FIG. 13, the spacers 44 are integrally formed with the semi-spherical clear plastic wall 36.

Although the invention has been described with reference to preferred embodiments, it will be appreciated by a person skilled in the art that the invention may be embodied in many other forms. For example, in other embodiments, the container 12 could be any suitable shape that defines an interior volume for the fluid 14 and mirror assembly 16. Also, in other embodiments, the orientating means 22 could be made from any material that is buoyant with the fluid 14. Also, in other embodiments, the reflecting surface is substantially convex or concave, depending on the focal length required by the user, and the orientating means 22 horizontally levels an outer circular periphery of the reflecting surface. The convex/concave reflecting surface is to accommodate users with different visions (e.g. short sighted and/or long sighted) and/or to vary the degree of magnification required. Also, in other embodiments, the device 10 may be used to simply perform self-examination of the eye of the user and not necessarily for the administration of eye-drops.

1. An eye-dropper positioning device including:
a container having at least a substantially transparent portion and defining an interior;
a fluid at least partially filling the interior of the container; and
a mirror assembly located in the interior of the container, the mirror assembly comprising:
at least one reflecting surface; and
an orientating means buoyantly supported by the fluid and configured to substantially face the at least one reflecting surface downwardly such that an image of an eye under the container is reflected by the at least one reflecting surface back to the eye through the substantially transparent portion of the container.

2. The eye-dropper positioning device according to claim 1, wherein the orientating means is located above the at least one reflecting surface.

3. The eye-dropper positioning device according to claim 1 or 2, the orientating means comprises a material buoyant with the fluid.

4. The eye-dropper positioning device according to claim 3, the material comprises cork.

5. The eye-dropper positioning device according to claim 1 or 2, the orientating means comprises an air-filled chamber.

6. The eye-dropper positioning device according to any one of the preceding claims, wherein the container is substantially shaped as a sphere.

7. The eye-dropper positioning device according to any one of the preceding claims, wherein the interior of the container and the mirror assembly each define a diameter, the diameter of the mirror assembly being less than the diameter of the interior.

8. The eye-dropper positioning device according to any one of the preceding claims, wherein the substantially transparent portion of the container and/or the fluid magnifies the image of the eye.

9. The eye-dropper positioning device according to any one of the preceding claims, wherein the substantially transparent portion of the container is formed from polycarbonate.

10. The eye-dropper positioning device according to any one of the preceding claims, wherein the substantially transparent portion of the container is formed from polypropylene.

11. The eye-dropper positioning device according to any one of the preceding claims, wherein the fluid is of a high viscosity.

12. The eye-dropper positioning device according to any one of the preceding claims, wherein the fluid comprises liquid silicone.

13. The eye-dropper positioning device according to any one of the preceding claims, wherein the fluid is substantially transparent.

14. The eye-dropper positioning device according to any one of the preceding claims, further comprising an attachment mechanism configured to allow attachment of a swivel connector to the container.

15. The eye-dropper positioning device according to any one of the preceding claims, wherein the at least one surface is substantially planar.

16. The eye-dropper positioning device according to any one of claims 1 to 14, wherein the at least one reflecting surface is substantially outwardly convex.

17. The eye-dropper positioning device according to any one of claims 1 to 14, wherein the at least one reflecting surface is substantially concave.

18. The eye-dropper positioning device according to any one of claims 15 to 17, wherein the orientating means is further configured to substantially horizontally level at least an outer periphery of the at least one surface.

19. A method for delivering an eye-drop from an eye-dropper to an eye, the method comprising:
positioning a mirror assembly having at least one reflecting surface above the eye;
facing the at least one reflecting surface downwardly such that an image of the eye is reflected back to the eye;
positioning an eye-dropper using the reflected image of the eye such that a nozzle of the eye-dropper is located between the at least one reflecting surface and the eye, and the nozzle is aimed at a desired portion of the eye; and
actuating the eye-dropper to deliver the eye-drop to the desired portion of the eye.

20. The method according to claim 19, wherein the mirror assembly comprises an orientating means, and wherein the step of facing the at least one reflecting surface downwardly comprises buoyantly supporting the orientating means of the mirror assembly in a fluid.

21. The method according to claim 20, wherein the orientating means comprises a buoyant material.

22. The method according to claim 20 or 21, wherein the fluid is of a high viscosity.

23. The method according to any one of claims 19 to 22, further comprising magnifying the reflected image of the eye prior to being reflected back to the eye.

24. The method according to any one of claims 19 to 23, wherein the desired portion is the pupil of the eye.

25. The method according to any one of the claims 19 to 23, wherein the desired portion is the white of the eye.

26. The method according to any one of claims 19 to 25, wherein the at least one reflecting surface is substantially planar.

27. The method according to any one of claims 19 to 25, wherein the at least one reflecting surface is substantially outwardly convex.

28. The method according to any one of claims 19 to 25, wherein the at least one reflecting surface is substantially concave.

29. The method according to any one of claims 26 to 28, wherein the step of facing the at least one reflecting surface downwardly comprises horizontally levelling at least an outer periphery of the at least one reflecting surface.

30. An eye examination device, including:
a container having at least a substantially transparent portion and defining an interior;
a fluid at least partially filling the interior of the container; and
a mirror assembly located in the interior of the container, the mirror assembly comprising:
at least one reflecting surface; and
an orientating means buoyantly supported by the fluid and configured to substantially face the at least one reflecting surface downwardly such that an image of an eye under the container is reflected by the at least one reflecting surface back to the eye through the substantially transparent portion of the container.
